(19) United States
(12) Patent Application Publication
(10) Pub. No.: US 2004/0261791 A1
(43) Pub. Date: Dec. 30, 2004

(54) Nasal dilator and method of nasal dilation

(76) Inventor: Richard C. Horian, Los Angeles, CA (US)

Correspondence Address: FULBRIGHT AND JAWORSKI L L P, PATENT DOCKETING 29TH FLOOR, 865 SOUTH FIGUEROA STREET, LOS ANGELES, CA, 900172576

(21) Appl. No.: 10/606,004

(22) Filed: Jun. 24, 2003

(51) Int. Cl. A61M 15/00; A61M 16/00

(52) U.S. Cl. 128/200.24; 128/206.11

(57) ABSTRACT

Nasal dilators comprise tubes having a length to width ratio of no more than unity. The tubes are right cylinders which are shown in circular, oblong and radiused cornered rectangular forms. One or more tabs can extend from the outer end of the tube, a rim may extend outwardly about the periphery of the inner end of the tube and a septum may diametrically bisect the hole within the tube. Dilator kits provide variable outside diameters for accommodating differences in nose structure. A member extending between two such tubes can be employed to provide a set for both nasal passages of the nose.

### BACKGROUND OF THE INVENTION

The field of invention is nasal dilation.

Snoring affects 45% of the population from time to time and an estimated 25% snore habitually. Snoring can also contribute to obstructive sleep apnea syndrome. OSAS, consists of frequent interruption of breathing during sleep, owing to intermittent collapse of the upper airway. Untreated, sleep apnea can cause high blood pressure and other cardiovascular disease, memory problems, weight gain, impotency, and headaches. Moreover, untreated sleep apnea may be responsible for job impairment and motor vehicle crashes. Most recently, studies have been conducted leading to evidence that “Sudden Infant Death Syndrome” may actually be caused by sleep apnea. Personal relationships are often damaged by partners snoring. Athletes can greatly improve performance by increased nasal breathing. Many can benefit by an improved nasal dilator.

There are basically three different types of nasal dilators employed over the years to help unblock nasal passages allowing air to pass more freely. External nasal dilators usually consist of an adhesive backed spring pad designed to be applied across the outside of the nose.

The other two types are internal insert nasal dilators designed to be placed inside the nasal cavity. The internal insert devices can further be classified into two basic types of design. Wire form types consist primarily of interconnected mesh forms or spring bars designed with the purpose of expanding the nasal canal wherever contacted by the form members. The wire form spring bar devices potentially have a disadvantage. The tissue inside the nasal canal is very delicate and sensitive. Any individual bar member contacting this delicate tissue with sufficient force to free the entire airway passage can potentially cause discomfort.

Tubular form types differ from the other types of nasal dilators in that the contacting wall surface inside the nasal cavity is contiguous. The entire surface area of the nasal canal is expanded by the contact area of the tube device. Pressure to expand the delicate nasal tissue is more evenly distributed by the uniform contact area derived by the contiguous wall of the tube. U.S. Pat. Nos. 1,256,188; 2,335,936; 2,569,743; 2,672,138; 5,665,104 and Des. 388,172 all teach variations of the internal insert tubular nasal dilator and show length to width ratios exceeding unity dictating potentially disadvantageous application.

Certain of the tubular form types are applied by being inserted deeply into the nasal cavity so that the bottom of the tube is basically flush with the bottom opening of the nostril. The human nose structure, illustrated in FIG. 1, around the immediate nostril opening is surrounded by the septum, intermediate crus, sesamoid cartilage and lower lateral cartilage-lateral crus. This area is generally elastic and fleshy with the exception of the lower lateral cartilage above the opening of the nostril. The lower lateral cartilage is connected only to the fleshy membrane around it. It is not connected directly to the rigid cartilage members above that give the rhinion area of the outer nose it's distinctive shape.

Above the lower lateral cartilage is the upper lateral cartilage separated only by the intranasal suture line connected to the rigid nasal bone above. Any tubular nasal dilator device designed to be inserted deeply enough to potentially affect the upper lateral cartilage has a different function. It is this area where many individuals suffer a collapse of the generally narrowing airway. However, it is this area that is also most sensitive to manipulation by an external device protruding within. Many individuals have a deviated septum or other anomaly that affects sensitivity to this area. It can be painful to have this area manipulated by direct force expansion.

Such elongate devices may not be inserted so deeply as to physically engage the upper lateral cartilage. Such tubes as illustrated in the prior art are of a length to width ratio to substantially protrude below the lower nostril area if not placed so deeply. It was found through testing that any tube device, or connecting members such as tie straps, that protrudes substantially below the nostril area can easily be dislodged or repositioned during sleep.

It is common during sleep to touch the face or affect the nose area with contact of the pillow during sleep movement. A device that protrudes substantially below the lower nostril area can potentially be dangerous. Sleep movement can be violent at times when sleep is troubled. It is possible to hit this area of the nose with sufficient force to drive the dilator further up the nasal canal than intended. This can cause pain and potentially damage. There is also an increased potential for damage if the worn device protrudes substantially below the nostril during sports activities.

### SUMMARY OF THE INVENTION

The present invention is directed to internal nasal dilation. Tubes having a length to width ratio of no more than 1 are placed in the nose.

In a first separate aspect of the present invention, the tube includes a rim at one end about the periphery of the tube. This rim is outwardly extending to assist in opening the nasal passage.

In a second separate aspect of the present invention, the tube includes a differential and compressive stress to strain ratio angularly about the cylindrical tube. Rotation of the tube then can result in increased spreading of the nasal passage after insertion of the tube. The angular differential may be provided by a septum extending diametrically across the tube.

In a third separate aspect of the present invention, the tube includes a differential in effective width in a nasal passage angularly about the cylindrical tube. Such differential can be provided by the tube being a noncircular cylinder such as an oblong cylinder or a radiused cornered rectangular cylinder. Again, rotation of the tube provides the differential width after placement within the nasal passage.

In a fourth separate aspect of the present invention, a nasal dilator kit of such tubes which are in substantially identical sets and varying across the normal range of nasal passage diameters includes intermediate tubes to provide proper fit within each nasal passage.

In a fifth separate aspect of the present invention, a process of nasal dilation includes selecting the appropriate sized tube and inserting that tube in the nasal passage not to extend into the passage to adjacent the upper lateral cartilage and not to extend substantially from the end of the nasal passage. Rotation of the tube to achieve further opening of the nasal passage may be additionally employed once the tube is appropriately inserted.

In a sixth separate aspect of the present invention, any of the foregoing aspects are contemplated to be employed in combination to further advantage.

### BRIEF DESCRIPTION OF THE DRAWINGS

FIG. 1 is a diagrammatic representation of a nose.

FIG. 2 is a perspective view of a nasal dilator.

FIG. 3 is a nose with the nasal dilator of FIG. 2 illustrated in perspective.

FIG. 4 is a perspective view of a nasal dilator kit.

FIG. 5 is a perspective view of a second embodiment of a nasal dilator.

FIG. 6 is a perspective view of a third embodiment of a nasal dilator.

FIG. 7 is a perspective view of a fourth embodiment of a nasal dilator.

FIG. 8 is a perspective view of a fifth embodiment of a nasal dilator.

FIG. 9 is a perspective view of a sixth embodiment of a nasal dilator.

FIG. 10 is a front view of a nose with the nasal dilator of FIG. 9 placed therein.

FIG. 11 is a perspective view of a seventh embodiment of a nasal dilator.

FIG. 12 is a perspective view of an eighth embodiment of a nasal dilator.

### DETAILED DESCRIPTION OF THE PREFERRED EMBODIMENTS

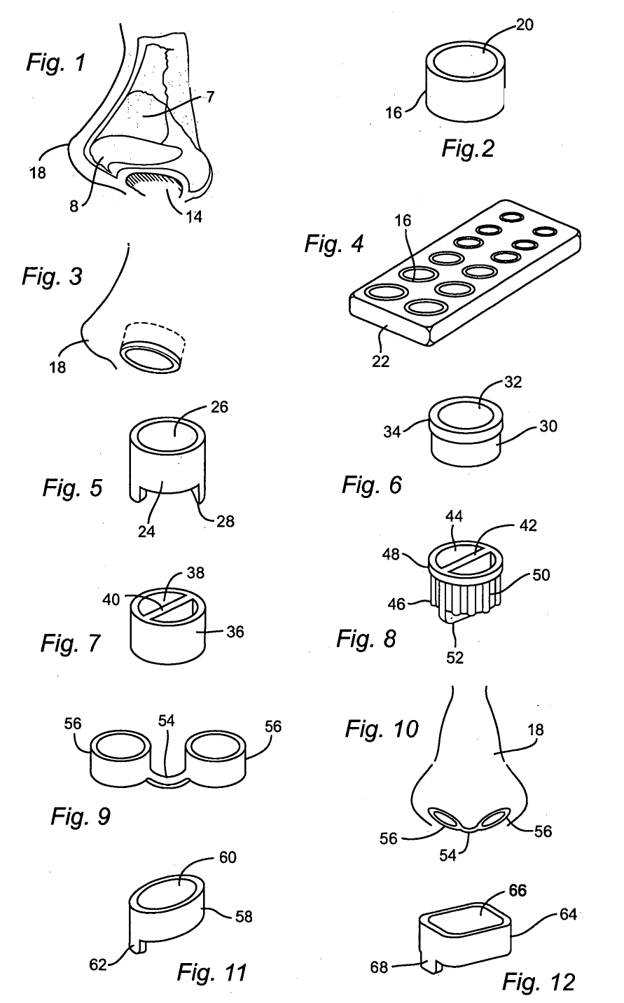

Turning in detail to the drawings, FIG. 1 illustrates a nose in a schematic view. Of relevance here is the upper lateral cartilage 10, the lower lateral cartridge 12 and the nasal passage 14.

FIG. 2 illustrates a minimal nasal dilator tube 16. The tube 16 has an outside dimension sized to be snugly received by normal nasal passages 14. The tube 16 might range from 0.375 inches o.d. to 0.75 inches o.d. for adults and 0.125 inches o.d. to 0.500 inches o.d. for children. The height of the tube 16 is such that the length to width ratio of the tube 16 does not exceed unity. The tube 16 is illustrated in place in a nose 18 in FIG. 3.

The tube 16 is made of a flexible elastomeric material. The thermal plastic elastomer family of plastics are appropriate for this use. Santoprene® manufactured by Advanced Elastomer Systems is one such elastomer that is nonhygroscopic as well as suitable for medical device applications. However, there are many other elastomerics suitable for use as the integral tube 16 such as natural rubbers, synthetic rubbers, thermoform elastomerics, thermoset elastomerics and combination blended materials.

The tube 16 is flexible with rounded edges to aid in placement and to avoid irritation in place. The tube 16 of FIG. 2 is shown to be a right circular cylinder. The wall thickness of the tube is preferably between 0.010 inches and 0.100 inches. The material of the tube 16 is preferably resilient at the foregoing thicknesses such that it will regain its shape even though it may be collapsed for insertion into the nasal passages

The relationship of the length to width of the tube 16 allows it to be positioned in a normal nose without extending into the nasal passage 14 so far as to be adjacent the upper lateral cartilage 10. At the same time, the tube 16 does not extend substantially from the nasal passage 14. Such placement is illustrated in FIG. 3. The tube 16 expands the region within which it is positioned in the nasal passage 14. The lower lateral cartilage 12 is able to move such that pressure on the delicate areas of the upper nasal passage will not be impacted by compression between the tube 16 and the lower lateral cartilage 12. Such would not be the case if the tube 16 extended into adjacent the upper lateral cartilage 10. Consequently, the ratio of length to width of the tube 16 being equal to or less than unity avoids the difficulties associated with insertion into adjacent the upper lateral cartilage 10.

At the same time, the placement of the tube 16 in the lower lateral area of the nostril cavity and its expansion of the soft tissue surrounding also acts to draw the soft tissue adjacent to the upper lateral cartilage 10 forwardly and outwardly to open up the nasal passage adjacent to the upper lateral cartilage 10, beyond where the tube 16 extends. Thus, a clear passage is created in the nasal passage 14 both adjacent the lower lateral cartilage 12 through the hole 20 in the nasal dilator tube 16 and past the upper lateral cartilage 10 through the influence of the tube 16 on the soft tissue in that area.

The length of the tube 16 also provides for the tube not extending substantially from the end of the nasal passage 14. This is advantageous because there is a tendency for the bedding or human contact to engage or otherwise interfere with tubing which extends from the nose. This can result in dislodging the tube from the nose or injuring the nasal soft tissue if abruptly impacted.

FIG. 4 illustrates a set of nasal dilators retained within a container 22. Dilators 16 are arranged as two sets for general retail sales. When offered as a kit in two sets, the tubes 16 are arranged in graduating size diameters in the appropriate range depending on application for the individual. Again, a practical kit for adults would include outside dimensions of tubes 16 of about 0.375 inches to 0.75 inches. The increments may be anywhere between 0.010 inches and 0.125 inches. With that range, a minimum of two tubes per set in two sets would be included within the container 22. The other embodiments addressed below could also be accommodated by the same or similar container 22.

FIG. 5 illustrates one variation on the tube 16 of FIG. 2. In the embodiment of FIG. 5, a right circular cylindrical tube 24 is shown to include a hole 26 therethrough and tabs 28. These one or more tabs 28 would extend outwardly of the nasal passage to a small extent for easy manual purchase for insertion and removal.

FIG. 6 illustrates yet another embodiment also employing a right circular cylindrical tube 30 with a hole 32 therethrough. A rim 34 extends outwardly about the periphery of the tube 30 at one end thereof. The tube 30 is intended to be placed in the nasal passage 14 with the rim 34 at the inside top edge. The soft tissue within the nasal passage 14 accommodates this rim 34, creating a impermanent set which helps retain the tube 30 in place. The rim 34 also increases the pressure a bit to further draw soft tissue adjacent the upper lateral cartilage 10 to promote opening of the nasal passage

FIG. 7 illustrates yet another embodiment with a nasal dilator tube 36 having a hole 38 therethrough. The tube 36 is again shown to be a right circular cylinder with a length to width ratio of no more than unity. A tube septum 40 extends diametrically across the hole 38 and is integrally formed with the tube 36. This septum 40 provides a differential compressive stress to strain ratio angularly about the tube 36. Compressing the tube parallel to the septum tube 40 is more difficult than compressing the tube perpendicularly to the septum 40. Thus, a maximum stress to strain ratio is experienced in alignment with the septum 40 while a minimum stress to strain ratio is perpendicular thereto.

The foregoing contemplates the tube septum 40 being sufficiently rigid to exhibit column strength. If the septum 40 is thin, the maximum stress to strain ratio may be accomplished at 90° to the tube septum 40 instead where the septum 40 acts in tension. This property may be thought of also as a differential effective width as the user perceives that the nasal dilator 36 is wider or narrower depending on its orientation given the stress to strain ratios.

Employment of the embodiment of FIG. 7 provides for insertion of the tube 36 into longitudinal position within the nasal passage 14. Rotation of the nasal dilator 36 then provides for increased soft tissue distortion in whichever way a greater opening requirement is desired, typically with the maximum resistance to compression being oriented laterally within the nose.

FIG. 8 illustrates a variation on the embodiment of FIG. 7. In FIG. 8, a septum 42 extends across the hole 44 of a nasal dilator tube 46. Again, the tube 46 is a right circular cylinder. A rim 48 as in the embodiment of FIG. 6 is also employed. The outside surface of the tube 46 includes longitudinally extending splines 50. These splines 50 provide surface height variations, in this instance, to resist rotation by helping to seat the device within the surrounding soft tissue. Bumps, grids and other variations or patterns may be employed instead. In addition to the splines 50, the septum 42 has an extended length exceeding the tube to form a protruding tab 52. This tab 52 extends from one end of the dilator 46. This tab 52 makes it more convenient to rotate, insert or retract the dilator relative to the nasal passage 14. Again, the main body of the tube 46 has a height to width ratio of no greater than unity. By locating the tab 52 centrally at one end of the tube septum 42, manual acquisition is easier without interference of the rim with the nasal passage 14.

FIG. 9 illustrates a member 54 extending between two tubes 56 as configured in FIG. 2. FIG. 10 illustrates the placement of the device of FIG. 9 in a nose. The member 54 is integrally formed with the two tubes 56 and is preferably quite flexible to admit of variation in the sizes and shapes of the nasal septum.

FIG. 11 illustrates a nasal dilator which is defined by a right oblong cylindrical tube 58 having a hole 60 and a tab 62. This oblong tube 58 has a differential effective width to provide a similar effect provided by the septum 40 in the embodiment of FIG. 7. A similar effect is provided by the nasal dilator of FIG. 12 having a tube 64 which is a radiused cornered rectangular cylinder having a hole 66 and a tab 68. This device of FIG. 12 provides some advantage in the rotation of the cylinder about its axis to change the effective width as placed in the nasal passage. The squared-off sides can act to stabilize the angular orientation of the dilator when one or more of the four sides is pressed against a substantially flat side of the nasal passage 14.

Thus, improved nasal dilators and methods for their use are disclosed. While embodiments and applications of this invention have been shown and described, it would be apparent to those skilled in the art that many more modifications are possible without departing from the inventive concepts herein. The invention, therefore is not to be restricted except in the spirit of the appended claims.

What is claimed is:

1. A nasal dilator kit for use in nasal passages, comprising
a plurality of at least four cylindrical tubes having a length to width ratio of no more than one, the plurality of tubes being in two substantially identical sets, each set including tubes varying substantially uniformly in major cross-sectional dimension from about approximately 0.375 inches o.d. to about approximately 0.75 inches o.d.

2. The nasal dilator kit of claim 1, each tube of the plurality of tubes including an outwardly extending rim at one end about the periphery of the tube.

3. The nasal dilator kit of claim 1, each tube of the plurality of tubes including a tab extending substantially parallel to the axial direction of the cylindrical tube from one end thereof.

4. The nasal dilator kit of claim 1, each tube of the plurality of tubes including a differential in effective width in a nasal passage angularly about the cylindrical tube.

5. The nasal dilator kit of claim 4, each tube including a maximum and minimum cross-sectional o.d.

6. The nasal dilator kit of claim 4, each tube including a differential in compressive stress to strain ratio angularly about the cylindrical tube.

7. The nasal dilator kit of claim 1, each tube of the plurality of tubes including a differential in compressive stress to strain ratio angularly about the cylindrical tube with the maximum ratio and minimum ratio being at about 90° to one another.

8. The nasal dilator kit of claim 7, each tube of the plurality of tubes including a tab extending substantially parallel to the axial direction of the cylindrical tube from one end thereof.

9. The nasal dilator kit of claim 7, each tube of the plurality of tubes being a right circular cylinder.

10. The nasal dilator kit of claim 7, each tube of the plurality of tubes including a diametrically extending septum.

11. The nasal dilator kit of claim 10, each septum including a tab extending substantially parallel to the axial direction of the cylindrical tube from one end thereof.

12. The nasal dilator kit of claim 1, the tubes each including an outside surface having surface height variations thereon.

13. The nasal dilator kit of claim 1, further comprising
a plurality of connecting members extending between tubes of like major cross-sectional dimension from each set.

14. A nasal dilator comprising
a cylindrical tube having a length to width ratio of no more than one, the tube including an outwardly extending rim at one end about the periphery of the tube.

15. A nasal dilator comprising
a cylindrical tube having a length to width ratio of no more than one, the tube including a differential in compressive stress to strain ratio angularly about the cylindrical tube with the maximum ratio and minimum ratio being at about 90° to one another.

16. The nasal dilator of claim 15, the tube including a tab extending substantially parallel to the axial direction of the cylindrical tube from one end thereof.

17. The nasal dilator of claim 15, the tube being a right, circular cylinder.

18. The nasal dilator of claim 15I the tube including a diametrically extending septum.

19. The nasal dilator of claim 18, the septum including a tab extending substantially parallel to the axial direction of the cylindrical tube from one end thereof.

20. A nasal dilator comprising
a cylindrical tube having a length to width ratio of no more than one, the tube including a differential in effective width in a nasal passage angularly about the cylindrical tube.

21. The nasal dilator of claim 20, the tube including a tab extending substantially parallel to the axial direction of the cylindrical tube from one end thereof.

22. The nasal dilator of claim 20, the tube being a right, non-circular cylinder.

23. The nasal dilator of claim 22, the tube being an oblong cylinder.

24. The nasal dilator of claim 22, the tube being a radiused cornered rectangular cylinder.

25. Nasal dilation comprising
selecting one from a plurality of tubes of different diameters each having a length to width ratio of no more than one;
inserting the one tube into a nasal passage such that the tube does not extend in the nasal passage to adjacent the upper lateral cartilage and does not extend substantially from the end of the nasal passage.

26. The nasal dilation of claim 25, each tube including a differential in effective width in a nasal passage angularly about the cylindrical tube, the nasal dilation further comprising
rotating the tube to achieve a desired maximum fit with the nasal passage.

27. The nasal dilation of claim 25, each tube including a differential in compressive stress to strain ratio angularly about the cylindrical tube with the maximum ratio and minimum ratio being at about 90° to one another, the nasal dilation further comprising
rotating the tube to achieve a desired fit with the nasal passage.
